(19) United States
(12) Patent Application Publication
(10) Pub. No.: US 2025/0311940 A1
(43) Pub. Date: Oct. 9, 2025

(54) METHOD AND DEVICE FOR AUTOMATICALLY COUNTING A USER'S BREATHING CYCLES WHEN PERFORMING BREATHING EXERCISES

(71) Applicant: David Blbulian, Larnaca (CY)

(72) Inventors: David Blbulian, Larnaca (CY); Anton Maltsev, Stavanger (NO); Vasily Morzhakov, Stavanger (NO); Anton Vasiliuk, Moscow (RU)

(21) Appl. No.: 18/629,709

(22) Filed: Apr. 8, 2024

(51) Int. Cl. A61B 5/08; A61B 5/00; A61B 7/00

(52) U.S. Cl. CPC A61B 5/0816; A61B 5/7264; A61B 5/7405; A61B 7/003

(57) ABSTRACT

An embodiment of a device for automatically counting a user's breathing cycles when the user performs breathing exercises includes at least: input/output devices as a microphone and a display, central and graphic processors, a memory with machine-readable instructions and an installed user application for automatically counting breathing cycles, as well as a database, a training sample, a machine learning algorithm, and a neural network.

### FIELD

Some embodiments relate to the use of neural network technology for automatically counting a user's breathing cycles when performing various types of breathing exercises. In some embodiments, the various types of breathing exercises include gymnastics or exercises implemented using the following techniques: pranayama from hatha yoga, breathing complexes using qigong, Tibetan yoga, transpersonal breathwork, and many other techniques.

### BACKGROUND

There are currently solutions that describe the use of neural networks in medical technologies that make it possible to diagnose the presence of a specific respiratory disease in a user by analyzing an incoming audio stream.

Such a solution was proposed by FLORIDA INST FOR HUMAN & MACHINE COGNITION INC in US 2021/0369136 A1. This solution provides a system based on artificial intelligence, which makes it possible to perform two types of research: a study to model the main mechanisms (hypercapnia, hypoxia) causing symptoms of COVID-19 infections, as well as a study to identify speech and breathing features characteristic of the occurrence of COVID-19 in patients. To do this, healthcare providers annotate the data in the audio stream obtained while the patient is reading a text out loud while exhaling, with special attention paid to the parameters of the user's inhalations and pauses in their speech. According to the authors of this solution, modeling changes in breathing and speech and subsequent acoustic analysis of users' breathing and speech will make it possible to identify distinctive features between normal and morbid or tense samples, which will then make it easier for healthcare providers to detect COVID-19 in users.

A similar solution was proposed by APPLICATIONS TECH APPTEK LLC in US 2021/0298711 A1. This solution provides a technology for the acoustic analysis of audio streams based on artificial intelligence for lung function auscultation and assessment and patients with mild forms of COVID-19. To do this, the authors of this solution developed a method for automatically segmenting and tagging audio recordings, which makes it possible, while the patient is reading the text, to determine the beginning and end times of inhalation, breath holding and exhalation, as well as coughing and other non-speech sounds (sneezing, coughing, etc.). To do this, healthcare providers annotate the data in the audio stream obtained while the patient reads the text aloud, with precise indication of the places of inhalation. The resulting sample is then used by the cloud service to analyze and assist healthcare providers in providing additional objective data on the respiratory status of patients.

Another solution is presented by HILL ROM SERVICES PTE LTD in US 2022/0061694 A1. This solution provides a system for monitoring the condition of a patient with a diagnosed respiratory disease, which is based on the analysis of audio data using a neural network. In particular, the authors of this solution found that the pitch, tone, rhythm, rate and volume of speech, as well as the duration of inhalation, duration of exhalation, or breathing rate during speech might indicate the presence of asthma, chronic obstructive pulmonary disease (COPD), bronchitis, emphysema, lung cancer, or other respiratory diseases. According to the authors, breaking the patient's speech into fragments and the automated analysis of its spectrogram makes it possible to diagnose the aforementioned respiratory diseases and simplify the process of making a specific diagnosis for the patient.

The developed solutions certainly have enormous potential in telemedicine, but they belong to a highly specialized branch of medical technologies that make it easier for healthcare providers to take decisions and make a diagnosis regarding a patient's respiratory disease. In addition, there is a large group of users who perform breathing exercises in order to obtain positive effects from them outside the medical industry.

Predominantly, these exercises involve performing a certain number of breathing cycles with different rate, depth, holds, number of rounds, and method of inhalation (through the nose/through the mouth). Usually, after a period of regularly performing breathing exercises, the degree of the effect decreases and the user has to find ways to restore the previous effectiveness of the exercise. One of the problems common to breathing exercises that reduces their effectiveness is the need to count the number of breathing cycles, which deflects part of the user's attention, distracts them from the breathing process itself, and negatively affects the final result and effect of the exercise. Available solutions in the medical technology market are difficult to use and commercially inaccessible for the average user, while solutions aimed at helping the user perform breathing exercises are typically pre-recorded or programmed videos or animations that the user can run and breathe in parallel with, synchronizing their pace with that set in the video or animation. This may be another reason for the decrease in effectiveness, because, in this case, it involves performing the exercise constantly at the same rhythm, dictated by a pre-recorded video or animation in the application.

Therefore, there is a demand for freeing the user who is performing breathing exercises from the need to count breathing cycles in their head, wasting their resources on this and losing concentration on breathing, and to adapt to the breathing rhythm provided by well-known commercial applications, which is probably not suitable for the user; and there is also a need to provide the user with the opportunity to easily adjust and change different parameters of the breathing exercise, including the rhythm, rate and time of the exercise, without worrying that they may lose count or not match the current count issued by the animation or video in the application.

Thus, the present invention is aimed to create a technical solution for automatically counting the user's breathing cycles when performing breathing exercises, making it easier for the user to perform the breathing exercise and focus all their attention on the exercises and sensations in the body, while providing the user with high flexibility in performing the breathing exercise, thereby contributing to a deeper and more pronounced effect from the breathing exercise.

### SUMMARY

One aspect of the present invention is a method for automatically counting a user's breathing cycles during breathing exercises, comprising:
the user entering a planned number of breathing cycles into the device;
the device receiving an audio stream while the user performs breathing exercises;
automatically counting the user's breathing cycles in the received audio stream by analyzing it using a neural network;
the device notifying the user when they reach the planned number of breathing cycles.

Another aspect of the present invention is a device for automatically counting a user's breathing cycles during breathing exercises that is configured to:
enter by the user a planned number of breathing cycles;
receive an audio stream;
automatically count the user's breathing cycles in the audio stream by analyzing it using a neural network;
notify the user when they reach the planned number of breathing cycles.

The neural network can be pre-trained on a sample including: a tagged audio stream with the beginning and end of the user's breathing cycles transformed into a one-dimensional sawtooth array, wherein the center of each inhalation and exhalation has a peak value. In addition, when creating a training sample, an audio stream can be used containing sounds other than those corresponding to the user performing breathing cycles. Taken together, this makes it possible to increase the accuracy of the neural network's analysis of the audio stream received while the user is performing breathing exercises and thereby increase the accuracy of counting breathing cycles while the user is performing the exercises.

During the neural network analysis, the audio stream can be divided into fragments, wherein the end of the previous fragment can overlap the beginning of the next one, and a neural network can process each fragment to obtain the probability of the center of inhalation or exhalation. In this case, the result can be integrated into a general plot, from which the number of peaks can be calculated. Taken together, this also further improves the accuracy of counting the user's breathing cycles during exercise.

While the user is performing breathing exercises, they can input a signal to hold their breath. A remote control device may be used for this purpose. To improve user convenience, a ring with a button may be provided as a remote control device.

The device for automatically counting the user's breathing cycles may contain a graphical interface that allows for entering the planned number of breathing cycles and may be configured to notify the user with an audio signal when recording that the user has reached the planned number of breathing cycles, which also simplifies counting the user's breathing cycles during exercise.

The device for automatically counting the user's breathing cycles may contain an electronic log and can be configured to automatically enter information about the parameters of the breathing cycles into the said log, which allows for the analysis of the results obtained when the user performs breathing exercises.

The advantages of aspects of the invention lie in simplifying and increasing the accuracy of counting the user's breathing cycles during breathing exercises due to the fact that the user's breathing cycles are automatically counted by an electronic device using a neural network pre-trained on a data sample specially prepared for this purpose, which all together increases the effectiveness of breathing exercises performed by the user.

### BRIEF DESCRIPTION OF DRAWINGS

FIG. 1 depicts a diagram of a user performing breathing exercises using a laptop device with an installed application for counting the user's breathing cycles.

FIG. 2 depicts a diagram of a user performing breathing exercises using an alternative device of a smartphone with an installed application for counting the user's breathing cycles.

FIG. 3 depicts a diagram of tagging a one-dimensional sawtooth data array for a machine learning algorithm used in the analysis of an audio stream by a neural network.

FIG. 4 depicts a flow diagram illustrating the process of a user performing breathing exercises using a device for automatically counting breathing cycles.

FIG. 5-9 depict a user interface of the application for automatically counting the user's breathing cycles.

### DETAILED DESCRIPTION

The present invention provides the user with a device for automatically counting breathing cycles, which is implemented while the user performs breathing exercises.

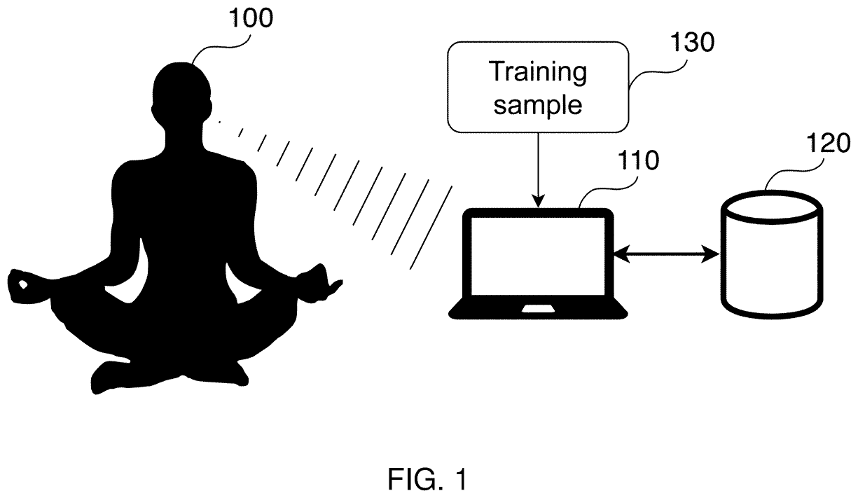

As shown in FIG. 1, a user 100 uses a device 110, provided as a laptop computer, desktop computer, tablet computer, or other computing device, the device 110 comprising at least: input/output devices as a microphone and a display, central and graphic processors, a memory with machine-readable instructions and an installed user application for automatically counting breathing cycles, a communication interface, a database 120, as well as a training sample 130, a machine learning algorithm, and a neural network.

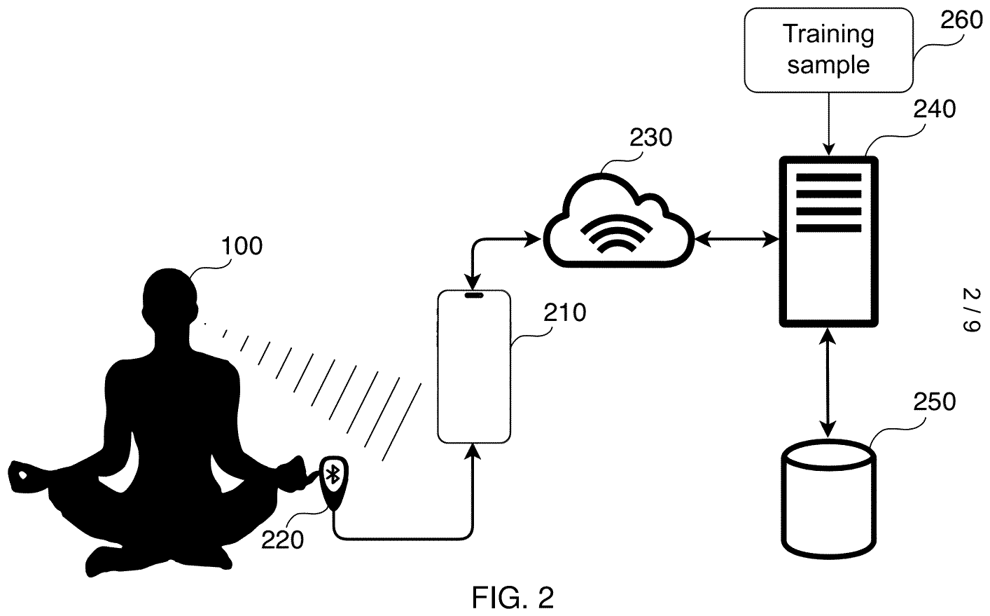

As shown in FIG. 2, the user 100 alternatively or additionally uses a device 210 such as a smartphone, personal digital assistant (PDA), or other personal or handheld computing device, the device 210 comprising at least: input/output devices as a microphone and a display, central and graphic processors, a memory with machine-readable instructions and an installed user application for automatically counting breathing cycles, and a communication interface through which a remote control (RC) device 220 provided as a ring with a button is connected to the smartphone. Note that it is also possible to use other remote control devices 220, such as a remote control console or smart watch, involving gestures pre-selected by the user 100. Furthermore, the smartphone is connected via the Internet 230 to a remote server 240, comprising at least a central processor, a memory with machine-readable instructions, a communication interface, a database 250, as well as a training sample 260, a machine learning algorithm, and a neural network.

Note that it is possible to use other devices that are capable of ensuring the required functionality of receiving an audio stream and its subsequent processing via a neural network as the device 110 or 210. Furthermore, note that the input device may also be a separate microphone or a microphone system that may be mounted on or adjacent to the body of the user 100 and connected to the devices 110 or 210 by any means of data transmission.

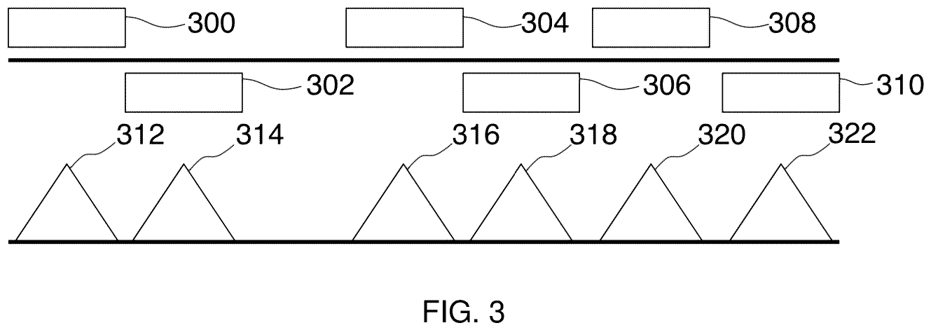

FIG. 3 shows a data tagging diagram for training samples 130 and 260 of the machine learning algorithm, wherein: blocks 300, 304, and 308 tag the beginning and end of the user's inhalations 312, 316, and 320, with their centers having a peak value equal to one, while blocks 302, 306, and 310 tag the beginning and end of the user's exhalations 314, 318 and 322, with their centers also having a peak value equal to one. Note that other data annotation schemes can be used to create training samples 130 or 260. In addition to the provided data, an audio stream containing sounds other than those corresponding to the user's performance of breathing cycles, such as footsteps, voices, clicks, etc., may be used when creating training samples 130 or 260.

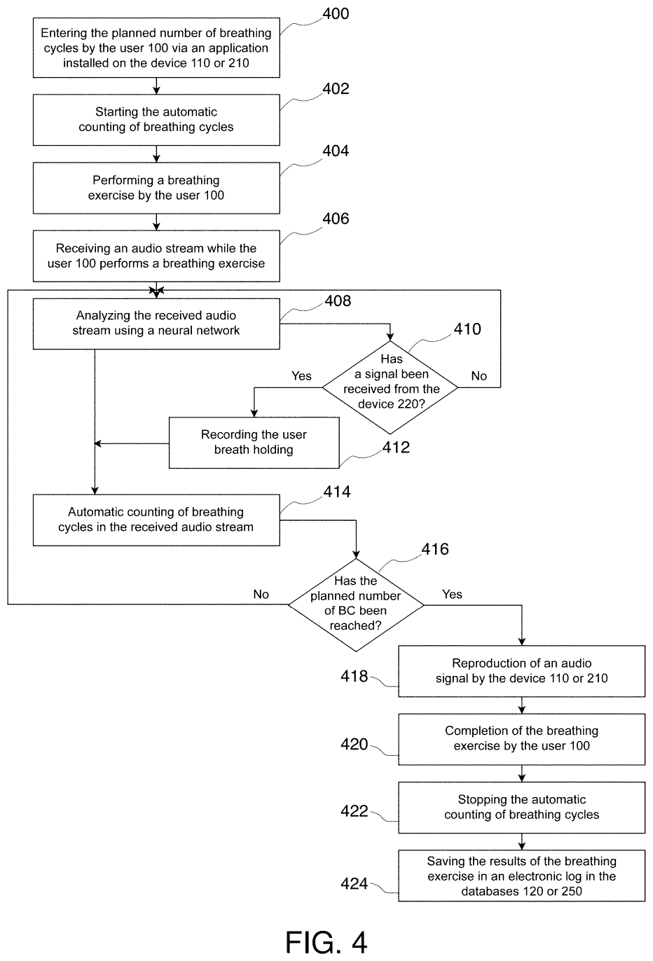

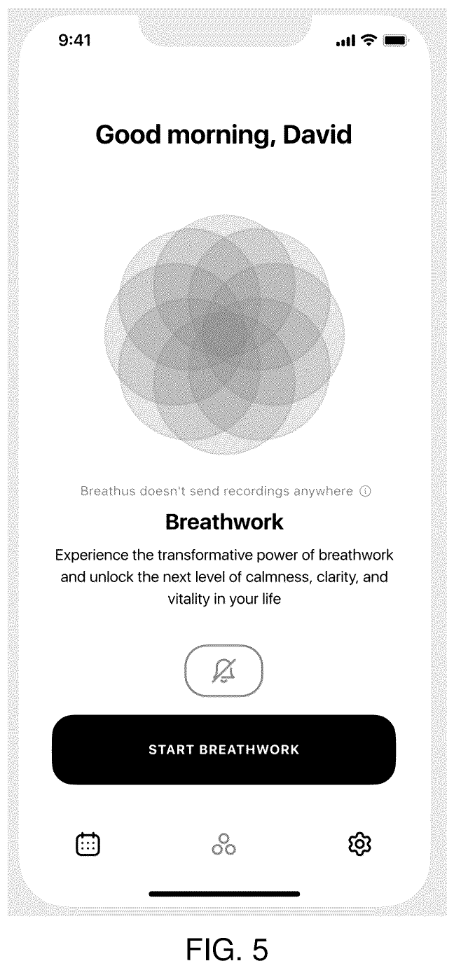

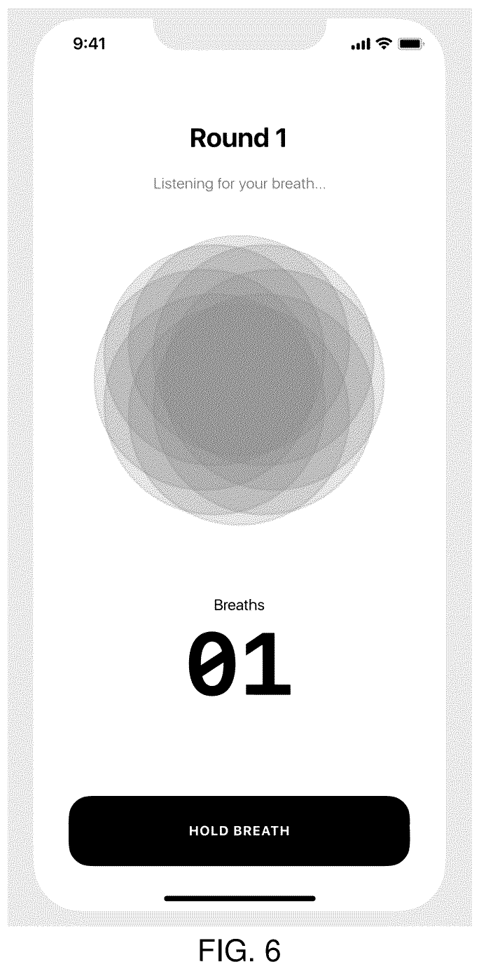

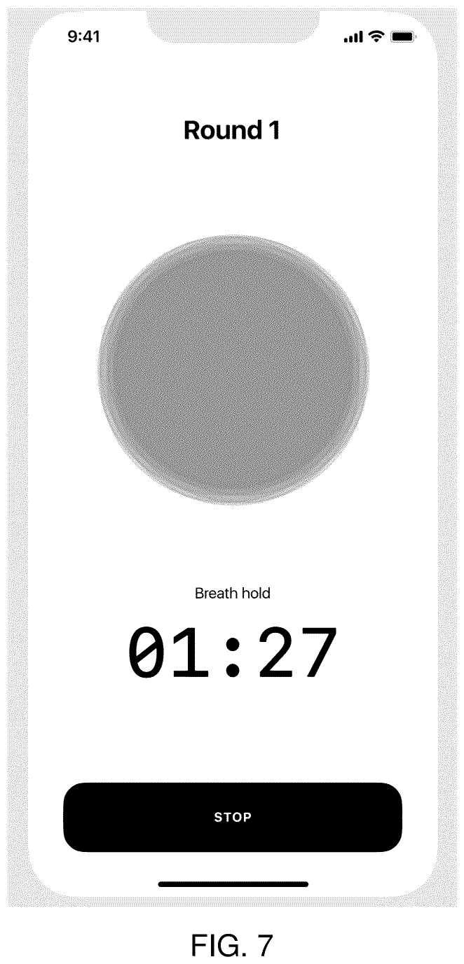

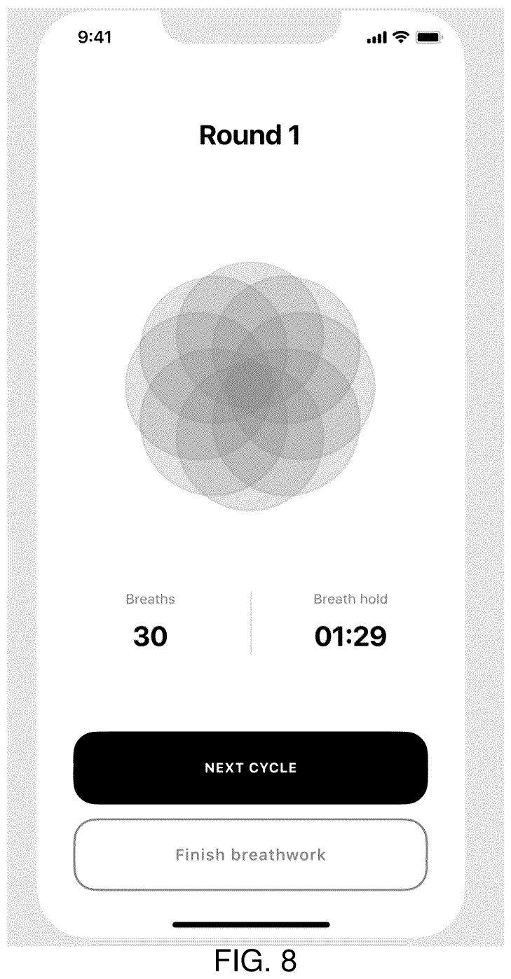

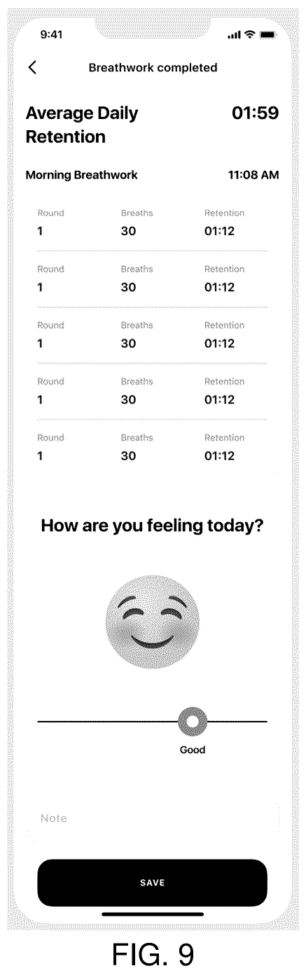

FIG. 4 shows a flow diagram illustrating the process of the user performing breathing exercises using a device 110 or 210 for automatically counting breathing cycles.

Step 400 describes preparing the user 100 to perform a breathing exercise, during which the user, via a user application installed on the device 110 or 210, enters the planned number of breathing cycles, for example, 30 units. A breathing cycle for the present invention is a repeating sequence of breathing phases that consists of inhalation and exhalation and may optionally include an inhalation hold and/or an exhalation hold. The user may utilize a graphical user application interface with, e.g., sliders, check boxes, and/or other means of entering data into the device 110 or 210 to enter the planned number of breathing cycles.

Step 402 describes the initiation of the automatic counting of breathing cycles, which can be performed either by the user 100 via the application or automatically by the device 110 or 210 itself in step 404.

Step 404 describes the user 100 performing a breathing exercise, which, in a particular embodiment, is implemented using the pranayama hatha yoga technique from hatha yoga. Note that as part of the present invention, breathing exercises can be implemented using other techniques. Automatic counting of breathing cycles can be started at this step by recognizing in the audio stream sounds that are characteristic of the breathing cycles of the user 100.

Step 406 describes the device 110 or 210 receiving an audio stream while the user 100 performs a breathing exercise, which is analyzed by the neural network at step 408.

Step 408 describes the neural network's analysis of the resulting audio stream. During the analysis, the audio stream is divided into fragments lasting approximately 20 sec, wherein the end of the previous fragment overlaps with the beginning of the next one, and a neural network processes each fragment to obtain the probability of the center of inhalation or exhalation, after which the result is integrated into an overall plot where the number of peaks is estimated. Note that when performing the breathing exercise, the user 100 may hold their breath, which may be accompanied by the user 100 engaging the remote control device 220 provided as a ring with a button at step 410. In this case, the device 210 at step 412 records the user 100 holding their breath until the user re-engages the remote control device 220, after which the analysis of the resulting stream continues. Note that breath holding may also be automatically recorded by devices 110 or 210.

Step 414 describes automatic counting of breathing cycles in the received audio stream, which, in a particular embodiment, is based on the estimate of the number of peaks obtained in step 408. At step 416, the number of breathing cycles completed by the user is compared with the planned number of breathing cycles entered before the start of the breathing exercise. If the number of performed breathing cycles has reached the planned number (e.g., 30 in an example), the device 110 or 210 at step 418 plays an audio signal or any other signal that can attract the attention of the user 100, vibrates, or provides some other output to attract the attention of the user. If the planned number of breathing cycles is not reached, the analysis of the received audio stream continues until it is reached and recorded.

At step 420, the user completes the breathing exercise or proceeds to repeat it or change the technique used.

Step 422 describes stopping the automatic counting of breathing cycles, which the user can do themselves or the device can do in automatic mode: for example, when recording a long absence of breathing cycles in the audio stream, wherein the results of the breathing exercise, including key parameters of the exercise-duration, rhythm, rate, breath holding time, etc.—at step 424 are saved in an electronic log placed in the databases 120 or 250 for subsequent analysis by the user.

The aforementioned description makes obvious a number of advantages of the present invention that free the user performing breathing exercises from the need to count breathing cycles in their head, wasting their resources on this and losing concentration on breathing, and to adapt to the breathing rhythm broadcast by well-known commercial applications, which is probably not suitable for the user; and there is also a need to provide the user with the opportunity to easily adjust and change different parameters of the breathing exercise, including the rhythm, rate and time of the exercise, without worrying that they may lose count, while, by saving the results, the user has the opportunity to analyze and select the most suitable parameters, combinations of parameters or exercise techniques.

Thus, the present invention allows the user to focus all their attention on performing the breathing exercises and sensations in the body and provides high flexibility in performing the breathing exercise, which contributes to a deeper and more pronounced effect.

Despite the features of the provided embodiments, they should not be construed as limiting the scope of embodiments of the invention, but solely as an illustration of some of them. Thus, the scope of all possible embodiments of the invention should be determined by the claims and not by the examples given in the description.

What is claimed is:

1. A method for automatically counting a user's breathing cycles during breathing exercises, comprising:
a) the user entering a planned number of breathing cycles into a device;
b) the device receiving an audio stream while the user performs breathing exercises;
c) the device automatically counting the user's breathing cycles in the received audio stream by analyzing it using a neural network;
d) the device notifying the user when the user reaches the planned number of breathing cycles.

2. The method of claim 1, wherein the neural network is pre-trained on a sample including at least one of:
i) a tagged audio stream with the beginning and end of the user's breathing cycles, transformed into a one-dimensional sawtooth array, wherein the center of each inhalation and exhalation has a peak value; or
ii) an audio stream containing sounds other than those corresponding to the user's breathing cycles.

3. The method of claim 2, wherein during analysis the audio stream is divided into fragments, wherein an end of a previous fragment overlaps with a beginning of a next one, and a neural network processes each fragment to obtain a probability of a center of inhalation or exhalation, and the result is integrated into an overall plot by which a number of peaks is estimated.

4. The method of claim 1, further comprising receiving a breath-holding signal while the user is performing a breathing exercise.

5. The method of claim 4, wherein a remote control device is used to input the breath-holding signal.

6. The method of claim 5, wherein the remote control device is a ring with a button.

7. The method of claim 1, further comprising playing an audio signal when the device determines that the user has reached the planned number of breathing cycles.

8. The method of claim 1, wherein information about parameters of the user's breathing cycles is automatically saved in an electronic log.

9. A device for automatically counting a user's breathing cycles during breathing exercises, configured to:
a) receive a planned number of breathing cycles from a user;
b) receive an audio stream;
c) automatically count the user's breathing cycles in the audio stream by analyzing it using a neural network;
d) notify the user when the user reaches the planned number of breathing cycles.

10. The device of claim 9, configured to receive a breath-holding signal from the user when automatically counting the user's breathing cycles.

11. The device of claim 10, to which a remote control device is connected allowing the user to input the breath-holding signal.

12. The device of claim 11, wherein the remote control device is a ring with a button.

13. The device of claim 9, comprising a graphical interface that allows for entering the planned number of breathing cycles and is configured to notify the user with an audio signal on detecting that the user has reached the planned number of breathing cycles.

14. The device of claim 9, comprising an electronic log, wherein the device is configured to automatically enter information about parameters of the user's breathing cycles into the log.
